(19) United States
(12) Patent Application Publication
(10) Pub. No.: US 2024/0093409 A1
(43) Pub. Date: Mar. 21, 2024

(54) METHOD FOR PRODUCING BONE REGENERATION MATERIAL HAVING COTTON-WOOL LIKE STRUCTURE

(71) Applicants: Nagoya Institute of Technology, Nagoya-Shi, Aichi (JP); ORTHOREBIRTH CO., LTD., Yokohama-shi, Kanagawa (JP)

(72) Inventors: Toshihiro Kasuga, Aichi (JP); Takashi Matsubara, Aichi (JP)

(73) Assignees: Nagoya Institute of Technology, Nagoya-Shi, Aichi (JP); ORTHOREBIRTH CO., LTD., Yokohama-shi, Kanagawa (JP)

(21) Appl. No.: 18/038,661

(22) PCT Filed: Nov. 18, 2021
(86) PCT No.: PCT/JP2021/042506

§ 371 Date: May 24, 2023

Related U.S. Application Data
(60) Provisional application No. 63/117,891, filed on Nov. 24, 2020.

(51) Int. Cl. D01D 5/00; A61L 27/46; A61L 27/54; D04H 1/728

(52) U.S. Cl. CPC D01D 5/0046; A61L 27/46; A61L 27/54; D04H 1/728; A61L 2300/104; A61L 2300/112; A61L 2300/404; A61L 2430/02; D10B 2401/063; D10B 2509/00

(57) ABSTRACT

A method for producing a cotton-wool like material for bone regeneration using a wet spinning method. 50-80 wt % of calcium salt particles and 50-20 wt % of PDLLGA resin are put into a mixing vessel, dissolved in acetone, and stirred to produce a spinning solution with a resin concentration of 10-20 wt % in which said calcium salt particles are dispersed. The produced spinning solution is filled in a syringe, and the spinning solution filled in the syringe is injected into a collector container filled with poor solvent by extruding the spinning solution from the discharge port of an injection needle having a predetermined diameter. The spinning solution injected into the poor solvent is solidified into fibers by interdiffusion of desorption of organic solvent and penetration of poor solvent in the poor solvent solution. The fibers solidified in the poor solvent are deposited in a floating state in the collector vessel without fiber-to-fiber adhesion and collected in a cotton-wool like shape.

### TECHNICAL FIELD

Present invention relates to a method for producing a bone regeneration material having a cotton-wool like structure formed of biodegradable fibers containing PLGA resin, and a bone regeneration material having a cotton-wool like structure produced by the method.

### BACKGROUND TECHNOLOGY

Bone regeneration materials are generally used in the form of blocks or granules. However, there is a demand for improvements in terms of moldability during surgery and concerns about movement and falling off from the target site. In order to meet the demand, polylactic acid with high rigidity is used as a matrix, mixed with inorganic fillers (β-phase-tricalcium phosphate, silicon-eluting calcium carbonate, etc.) and made into fibers by electrospinning (ES).

Inventors of the present invention succeeded to deposit biodegradable fibers emitted from the nozzle of ES apparatus in a collector container filled with ethanol, and collected and dried the fibers floating in the ethanol liquid to form cotton-wool like structure (U.S. Pat. No. 8,853,298). The cotton-wool like bone regeneration material is clinically superior because it can easily adapt to any shape of the affected area during surgery.

Recently PLGA has been used as a matrix resin for biodegradable fibers instead of polylactic acid. PLGA has higher bioabsorbability than polylactic acid, and is an excellent biodegradable resin whose safety has been approved by FDA. Therefore, using PLGA as a matrix resin, it is combined with inorganic fillers (β-phase tricalcium phosphate, calcium carbonate, etc.) and fiberized by electrospinning (ES).

PLGA is synthesized by copolymerizing lactic acid and glycolic acid, and the biodegradability can be controlled by adjusting the ratio of lactic acid and glycolic acid. Between PLGA (85:15) made of 85% lactic acid and 15% glycolic acid and PLGA (75:25) made of 75% lactic acid and 25% glycolic acid, the latter PLGA (75:25) has higher degradability.

On the other hand, lactic acid of polylactic acid may have a crystalline L-isomer and an amorphous D-isomer, which is an optical isomer. PDLLA containing D isomer is more difficult to crystallize and easier to degrade than PLLA which does not contain D isomer. Therefore, by copolymerizing PDLLA containing the D-isomer and PGA, it is possible to synthesize PDLLGA which has significantly higher degradability than PLGA (PLLGA) which does not contain D-isomer.

### Problems to be Solved by the Invention

Recently a method of producing a highly bioabsorbable bone regeneration material using a composite biodegradable fiber containing a PLGA resin and a calcium salt particle filler has been developed. Inventors of the present invention previously succeeded to produce biodegradable fibers containing PLLGA resin using ES process (U.S. Pat. No. 6,251,462). However, in order to dissolve PLLGA to produce spinning solution of ES, it is necessary to use a highly soluble chlorinated solvent (e.g., chloroform). From the safety point of view, it is not desirable to use chlorinated solvent which has strong toxicity in the production of bone regeneration material that will be implanted in a human body. On the other hand, PDLLGA is easily soluble in solvents and does not require use of chlorinated solvent. It can be dissolved in non-chlorinated solvent (eg. acetone). However, since PDLLGA has a lower molecular weight than PLLGA, it is difficult to maintain fiber form when ES method that requires application of a high voltage is used.

In addition, a bone regeneration material that is implanted in a human body are exposed to the risk of bacterial infection after implantation surgery. Therefore, it is desirable that the material itself has an antibacterial property.

### Means to Solve the Problem

As a result of intensive studies to solve the above problems, inventors of the present invention succeeded in spinning composite biodegradable fibers containing calcium salt particles in PDLLGA resin and forming the fibers in a cotton-wool like structure by using an improved wet spinning method.

The inventors of the present invention reached the invention of a method of using an improved wet spinning process to produce a cotton-wool like bone regeneration material, the method comprising:
preparing a spinning solution with a resin concentration of 10-20 wt % in which calcium salt particles are dispersed by mixing calcium salt particles of 50-80 wt % and PDLLGA resin of 50-20 wt % in a mixing container, and dissolving the mixture in acetone and stirring the dissolved mixture, and
filling the spinning solution thus prepared in a syringe,
injecting the spinning solution filled in the syringe into a collector container filled with poor solvent by extruding the spinning solution from the outlet of the injection needle having a predetermined diameter, wherein
the spinning solution injected into the poor solvent is solidified into fibers by interdiffusion of desorption of organic solvent and penetration of poor solvent, and the fibers solidified in the poor solvent are collected in cotton-wool like form by floating and depositing in the collector container without having adhesion between the fibers.

The inventors of the present invention further reached the invention of a cotton-wool-like bone regenerating material produced by using an improved wet spinning process, the cotton-wool like bone regenerating material is produced by the process of:
preparing a spinning solution with a resin concentration of 10-20 wt % in which calcium salt particles are dispersed by mixing calcium salt particles of 50-80 wt % and PDLLGA resin of 50-20 wt % in a mixing container, and dissolving the mixture in acetone and stirring the dissolved mixture, and
filling the spinning solution thus prepared in a syringe,
injecting the spinning solution filled in the syringe into a collector container filled with poor solvent by extruding the spinning solution from the outlet of an injection needle having a predetermined diameter of the syringe, wherein
the spinning solution injected into the poor solvent is solidified into fibers by interdiffusion of desorption of organic solvent and penetration of poor solvent, and the fibers solidified in the poor solvent are collected in cotton-wool like form by floating and depositing in the collector container without having adhesion between the fibers.

Preferably, the calcium salt particles are calcium phosphate particles, more preferably β-TCP particles. Silver-containing β-TCP is useful because of its antimicrobial properties.

Preferably the poor solvent is ethanol.

Preferably the poor solvent is water. If the water contains chlorine, it may react with silver contained in β-TCP to form AgCl. Therefore, it is preferable that the water is pure and chlorine-free.

Preferably the PDLLGA fibers contain 50-80 wt % of calcium salt particles, more preferably 60-70 wt %. In the wet spinning method, a spinning solution containing a large amount of calcium salt particles can be easily prepared since the fiber is spun by extruding a spinning solution prepared by mixing resin and filler particles and dissolving them in an organic solvent. In ES, because a slurry with low viscosity is used during spinning, the filler particles needs to be highly dispersed which requires a special process (e.g., kneading) to uniformly disperse a large amount of filler particles in the solution. Wet spinning does not require such a special process because it uses a slurry with a higher viscosity than that of ES. This is because the polymer solution that fills the space between the particles is less fluidic, agglomeration of particles can be avoided.

Preferably, calcium phosphate particles are used as calcium salt particles. More preferably β-TCP particles are used as calcium salt particles. In contact with body fluids, PDLLGA is degraded to release β-TCP particles. And further the β-TCP is dissolved, eluting calcium ions and phosphorus ions so that bone formation through bone resorption/replacement is promoted.

Preferably β-TCP particles synthesized by incorporating silver ions in the crystal lattice of β-TCP is used. As the β-TCP particles released from the PDLLGA fiber are dissolved, silver ions that are incorporated in the β-TCP are eluted from the PDLLGA fibers, resulting in antimicrobial activity.

Preferably resin concentration of the spinning solution is adjusted to be 10-20 wt %. Unlike ES method, wet spinning method discharges spinning solution through a nozzle by simply extruding it out, so the resin concentration of the spinning solution can be set relatively freely according to the extrusion rate and fiber thickness.

### Advantage of the Invention

The cotton-wool like bone regeneration material made of PDLLGA fibers produced by the wet spinning method of the present invention has high bioabsorbability and excellent flexibility making it suitable for use as a bone regeneration material in the dental field as well as in spine treatment.

In the present invention, because acetone is used instead of chloroform as the organic solvent for the preparation of the spinning solution, the material for bone regeneration produced by the present invention is safe.

PDLLGA fibers produced by the wet-spinning method of the invention have fewer pores on the fiber surface, a denser cross-sectional structure, and shape of the fiber is better maintained than the fibers spun by ES.

Unlike the ES method, because the wet spinning method of the present invention extrudes the spinning solution from the syringe to the outlet by applying physical force, there is a high degree of freedom regarding the content of filler particles in the spinning solution. By containing calcium phosphate of 50 wt %, more preferably 60 wt %, and even more preferably 70 wt %, the particles form an uneven structure on the fiber surface. The fiber surface having an uneven structure is suitable for cell adhesion.

In the cotton-wool like bone regeneration material made of PDLLGA fibers prepared by the wet spinning method of the present invention, PDLLGA is dissolved in the body after implantation in the body so that pH is decreased locally, creating an acidic environment. As a result, β-TCP is dissolved in the acidic environment, gradually releasing trace amount of calcium ions and phosphate ions, contributing to the promotion of bone formation.

When PDLLGA fibers produced by the wet spinning method of the present invention contain silver ion incorporated in β-TCP particles as fillers, PDLLGA is degraded in the body and pH is decreased locally creating acidic environment. As a result, β-TCP fillers are dissolved in the acidic environment, resulting in gradually releasing silver ions that are incorporated in β-TCP so that antimicrobial property is achieved. This enables the combination of PDLLGA fibers produced by the wet spinning method and silver ion incorporated in β-TCP particles to achieve the antimicrobial property in the late postoperative period after implantation of the bone regeneration material in the body.

In the wet spinning method of the present invention, because acetone used as an organic solvent does not contain chlorine, it does not produce AgCl when it comes into contact with silver. As a result, the Ag ions solidly dissolved in β-TCP do not become AgCl, but exist as Ag ions, thus demonstrating the antimicrobial properties of Ag ions. In addition, AgCl does not form and turn black when exposed to light.

### BRIEF DESCRIPTION OF DRAWINGS

FIG. 1 shows a schematic diagram of the wet spinning method of this invention when ethanol is used as a poor solvent.

FIG. 2 shows a sample material collected from poor solvent in the wet spinning method (using ethanol as poor solvent) in an embodiment of the present invention.

FIG. 3 shows the wet spinning method in an embodiment of the present invention when water is used as a poor solvent.

FIG. 4 shows a cotton-wool like bone regeneration material produced by using the wet spinning method (using water as a poor solvent) in this invention example.

FIG. 5 is an SEM photograph showing the surface topography of biodegradable fibers spun in the wet spinning method (using water as the poor solvent) in an embodiment of the present invention.

FIG. 6 shows a SEM photograph showing the surface irregularities of the spun biodegradable fiber in the wet spinning method (using water as the poor solvent) in this example.

FIG. 7 shows osteoblast adhesion to biodegradable fibers as a result of culturing osteoblasts for 6 hours, 1 day, and 3 days using cotton-wool like bone regeneration materials produced by using the wet spinning method (ethanol was used as a poor solvent) of an embodiment of the present invention.

FIG. 8 shows cells grow rapidly and steadily after 1 day of cell adhesion using cotton-wool like bone regeneration materials produced by using the wet spinning method (using ethanol as a poor solvent) in an embodiment of the present invention.

### EMBODIMENTS OF THE PRESENT INVENTION

Hereinafter, embodiment of the present invention is described in detail with reference to the drawings.

### Definition

<PLLGA Resin>

In the present invention, PLLGA resin refers to PLGA resin synthesized by copolymerization of lactic acid and glycolic acid containing only L-isomer. The polymerization ratio of 85:15 PLLA to PGA is called PLLGA(85:15) and the polymerization ratio of 75:25 PLLA to PGA is called PLLGA(75:25). Degradation of PLLGA can be enhanced by increasing the ratio of PGA. To dissolve PLLGA in a solvent, a chlorinated solvent such as chloroform must be used.

<PDLLGA Resin>

In the present invention, PDLLGA resin refers to PLGA resin synthesized by copolymerization of lactic acid containing D isomer and L isomer and glycolic acid. Lactic acid that is used to synthesize PLGA has a crystalline L-isomer and its optical isomer, amorphous D-isomer. PLA includes poly(L-lactic acid) (PLLA), which is composed of only the L-isomer, and poly(D-lactic acid) (PDLLA), which contains both L-isomer and D-isomer. It is possible to control the degradability of PDLLGA by changing the polymerization ratio of PDLLA to PGA. In the present invention, the amount of D-isomer in PDLLGA resin is sufficient to make the resin degradable and dissolvable in acetone by including D-isomer.

<Wet Spinning Method>

In this invention, wet spinning method refers to a method of solidifying spinning solution into fiber form by desorption of organic solvent and penetration of poor solvent. The choice of organic solvent and poor solvent affects the speed of polymer solidification and desorption/penetration of solvent. Balance of the speed of this desorption/penetration determines the form of the resulting fiber. The wet spinning method used in the present invention is modified and the conditions are set to fiberize PDLLGA resin containing calcium phosphate particles and form a cotton-wool like shape.

<Organic Solvent>

In the present invention, organic solvent is used to dissolve mixtures of PDLLGA resin and calcium phosphate particles. Chlorinated organic solvents such as chloroform have excellent solubility but are toxic. Acetone is inferior to chloroform in terms of solubility, but it is safe for living organisms because it does not contain chlorine. Since the PDLLGA resin used in the present invention is easily dissolved in a solvent, a safe non-chlorinated solvent such as acetone can be used without the need to use chloroform or other toxic chlorinated organic solvent.

<Poor Solvent>

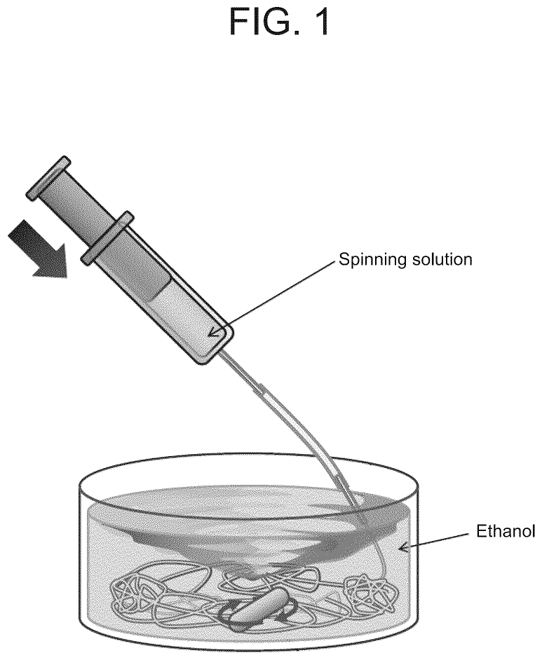

In the present invention, poor solvent is used in the coagulation bath as the solvent that does not dissolve PDLLGA resin. It is used to collect biodegradable fibers in a cotton-wool like form. Scholarly, poor solvent is said to be a poor solvent for this solute when the solute-solvent interaction (free energy) is less than the arithmetic mean of the solute-solute and solvent-solvent interactions in a particular substance-solvent system in terms of the theory. In the present invention, poor solvent is selected by taking into account the balance of desorption and penetration between the organic solvent and poor solvent. In the present invention, ethanol or water, in which PDLLGA is insoluble, can be used suitably as a poor solvent.
In a case that ethanol is used as a poor solvent, spinning solution can be made into fibers by stirring ethanol in a collector container and stretching the fibers by the flow of poor solvent produced by stirring, as shown in FIG. 1. In this case, Hansen solubility parameter of ethanol is 26.5 δ [(MPa)^{1/2}]) and that of acetone is 20.0 [(MPa)^{1/2}]), and the degree of difference between the two is 6.5 [(MPa)^{1/2}].
In a case that water is used as a poor solvent, when the spinning solution is extruded from the nozzle, the extruded spinning solution is fiberized and floated and deposited in the collector container. In this case, Hansen solubility parameter of water is 47.8 δ [(MPa)^{1/2}] and that of acetone is 20.0 δ [(MPa)^{1/2}]), and the degree of difference between the two is 27.8 δ [(MPa)^{1/2}].
Since degree of difference of Hansen solubility parameter of water from that of acetone is considerably greater than the difference of ethanol from acetone, the rate at which acetone is desorbed from the fiber is much faster than when ethanol is used as the poor solvent. As a result, the spinning solution extruded from the nozzle rapidly is fiberized in water, so there is no need to stretch the fibers by stirring the water to make the spinning solution fibrous.

<Silver Ion Solid Soluted β-Phase Tricalcium Phosphate>

In an embodiment of the present invention, silver ion incorporated β-phase tricalcium phosphate refers to a β-phase tricalcium phosphate in which the calcium sites in the crystal lattice of β-phase tricalcium phosphate are substituted by Ag+ ion.
Silver ion incorporated β-phase tricalcium phosphate can be prepared using the ultrasonic spray pyrolysis method. The ultrasonic spray pyrolysis method is one of the methods for synthesizing ceramic raw material powders. A sample solution is atomized by ultrasonic waves, and the droplets are introduced into a heated electric furnace to instantly remove solvent from the droplets, deposit salt, and cause pyrolysis to obtain powder (fine particles) with the desired chemical composition. Details are disclosed in JP-A2020-130417.

### Example 1 (Using Ethanol as a Poor Solvent)

The following materials and equipment were used
β-phase tricalcium phosphate (Ca₃ (PO)₄₂): Taihei Chemical Industry Co. β-TCP-100.

Particle size of 1.7 mm or less was pulverized to about 4 μm (β-TCP milled product).

### PDLLGA: PDLLA:PGA (75:25) (PURASORB PDLG7507, Corbion Purac)

Ethanol: Kishida Chemical first grade, purity 99.5%.
Acetone: Wako Pure Chemicals Reagent special grade purity 99.5+%.
Size of the extrusion opening of the injection needle for spinning solution extrusion: 27 G (inner diameter 0.2 mm, outer diameter 0.4 mm)
The container is a cylindrical vessel with a diameter of 15 cm and a height of 7.5 cm, and was stirred with a magnetic stirrer using a 5 cm long stirrer (see FIG. 1). (See FIG. 1).

### 1. Preparation of Spinning Solution

β-TCP and PDLLGA were mixed in a 7:3 weight ratio, dissolved in acetone, and mixed overnight to prepare a spinning solution with a polymer concentration of 17%.

### 2. Spinning Conditions

Extrusion speed 0.75 ml/h, stirring speed 200 rpm

### 3. Collection of Cotton-Wool Like Material

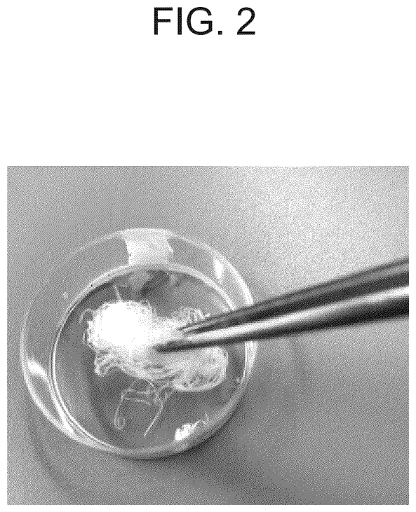

After wet spinning, the fibers were washed with ethanol and held overnight in ethanol to further remove the solvent. The ethanol was then removed with an absorbent sheet, and the cotton-wool like material was dried at room temperature while unraveling to obtain cotton-wool like Sample 1 (see FIG. 2).

### Example 2 (Using Water as a Poor Solvent)

The following materials and equipment were used
β-phase tricalcium phosphate (Ca₃(PO)₄₂): Taihei Chemical Industry Co. β-TCP-100. Particle size of 1.7 mm or less was ground to about 4 μm (β-TCP milled product).

### PDLLGA: PDLLA:PGA (75:25) (PURASORB PDLG7507, Corbion Purac)

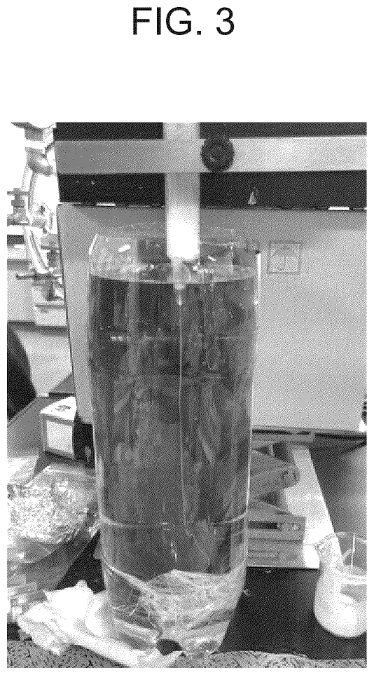

Pure water
Acetone: Wako Pure Chemicals Reagent special grade purity 99.5+%.
Size of the extrusion port of the injection needle for spinning solution extrusion: 33 G (inner diameter 0.07 mm, outer diameter 0.20 mm)
Poor solvent container: A cylindrical container with a diameter of 9 cm and a height of 25 cm was used (see FIG. 3).

### 1. Preparation of Spinning Solution

β-TCP and PDLLGA were mixed in a 7:3 weight ratio, dissolved in acetone, and mixed overnight to prepare a spinning solution with a polymer concentration of 17%.

### 2. Spinning Conditions

Extrusion speed 0.6 ml/h

### 3. Collection of Cotton Shapes

The solvent acetone is replaced with water and is removed from the fiber. However, because its specific gravity is smaller than that of water, it does not accumulate at the bottom of the container but floats near the top. As a result, even after conducting spinning for a long time, the acetone does not cause the fibers to stick to each other again, and long strokes of fiber are produced (see FIG. 3). In contrast, when ethanol is used as a poor solvent, the specific gravity of acetone and ethanol is almost the same, so when the fiber is spun in ethanol, the acetone that has been removed mixes with the ethanol and floats in the ethanol. As a result, when fibers are spun for a long time in the wet spinning method, the diluted acetone makes the fibers easily stick to each other, and the dried fibers become a lumpy mass.

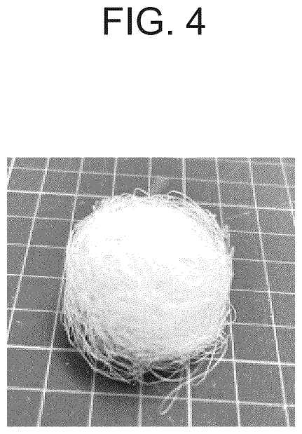

After wet spinning, the fiber is washed with ethanol and kept in ethanol overnight to further remove the solvent. The ethanol is then removed with an absorbent sheet, and the cotton-wool like material is dried at room temperature while unraveling to obtain cotton-wool like sample 2 (see FIG. 4).

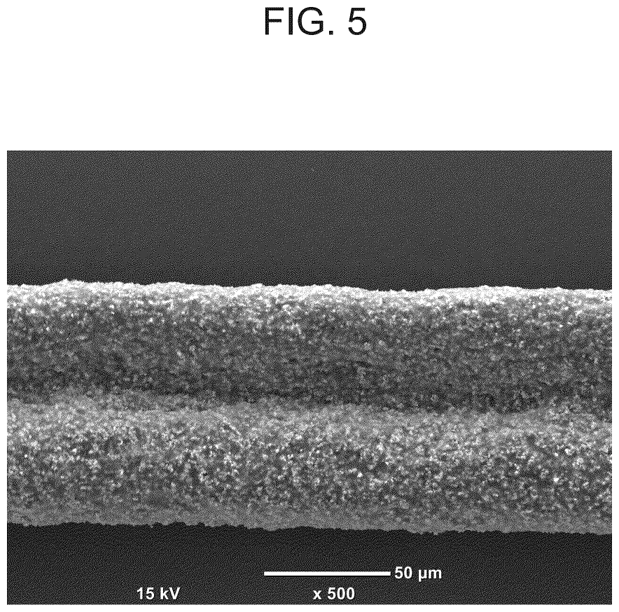

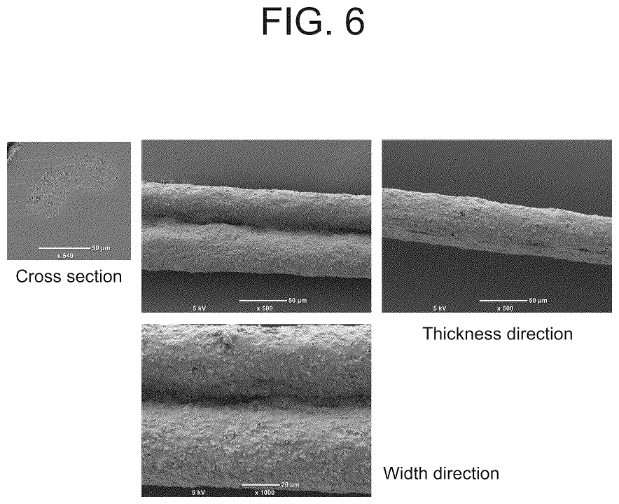

FIG. 5 shows a SEM photograph of β-TCP/PDLLGA fibers produced using water as the poor solvent. It is observed that filler particles are exposed on the surface of the fiber, forming an uneven topography.
FIG. 6 shows an SEM photograph of β-TCP/PDLLGA fibers produced using water as the poor solvent. The β-TCP/PDLLGA fibers are approximately 80-100 μm wide and 40-50 μm thick, with a flattened cross section with a dent on one side. According to the inventor's knowledge, the reason for this shape of the fiber is that a flow called “Kalman vortex” is generated when the fiber flows out of the syringe, and the center of the fiber is dented by this flow.

### 4. Experiment of Cell Adhesion on Cotton-Wool Like Material

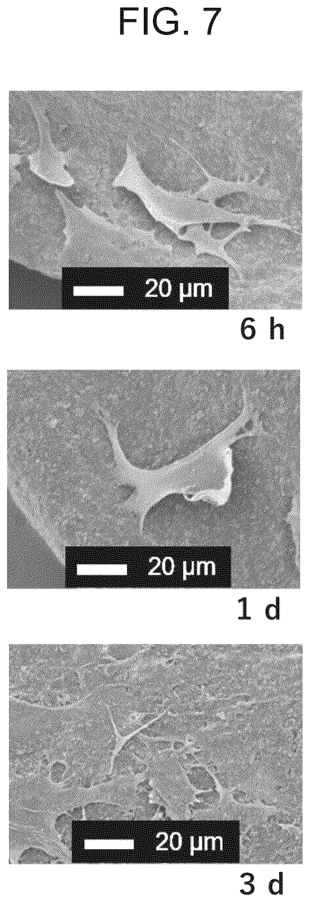

Wells were filled with 1 ml of normal medium and 0.5 ml of suspension (2.4×10⁵ cells/ml) of mouse-derived osteoblast-like cells (MC3T3-E1) after sample 1 was blended into the medium and cultured in an incubator for 6 hours, 1 day and 3 days (CO₂ concentration 5%, 37° C.). The adhesion of cells on the fibers constituting sample 1 was then observed using a scanning electron microscope. As a result of the experiment, it was observed that some cells began to adhere to the fiber surface by 1 day, and that they adhered and proliferated until they almost covered the surface in 3 days (see FIG. 7).
Wells were filled with 1 ml of normal medium and 0.5 ml of suspension (2.4×10⁵ cells/ml) of mouse-derived osteoblast-like cells (MC3T3-E1) after sample 1 was blended into the medium and cultured in an incubator for 6 hours, 1 day and 3 days (CO₂ concentration 5%, 37° C.).

AlamarBlue® Cell Viability Reagent (Thermo Fisher Scientific, here abbreviated as ABCVR) was added to normal medium to make ABCVR solution (normal medium:ABCVR=10:1 wt %). After transferring the medium from each incubated well to a centrifuge tube, 2.0 ml of ABCVR solution was added and kept in an incubator (CO₂ concentration: 5%, 37° C.) for 4 h to react. From the solution, 80 μl was taken and transferred to a black-bottomed 96-well plate for measurement. The fluorescence intensity was then measured using a multimode plate reader (Perkin Elmer Life & Analytical Sciences, EnSpire) (excitation wavelength: 540 nm, fluorescence wavelength: 590 nm). The fluorescence intensity at 6 hours was then compared with the premature decline intensity, which was set to 1, to evaluate the metabolic activity of the cells, i.e., to determine proliferative potential.

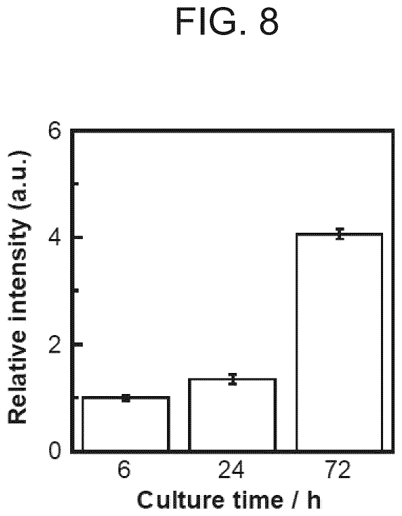

The results clearly showed rapid and steady growth after 1 day of cell adhesion (see FIG. 8).

The experimental results confirmed that the cotton-wool like bone regeneration material consisting of thick β-TCP/PDLLGA fibers spun by the wet spinning method of the present invention showed high proliferative potential in the osteoblast culture test.

1. A method of producing a cotton-wool like bone regeneration material by using a wet spinning process, the method comprising:
preparing a spinning solution with a resin concentration of 10-20 wt % in which
calcium salt particles are dispersed by mixing the calcium salt particles of 50-80 wt % and PDLLGA resin of 50-20 wt % in a mixing container to form a mixture, and
dissolving the mixture in acetone to form a dissolved mixture and stirring the dissolved mixture,
and
filling the spinning solution thus prepared in a syringe,
injecting the spinning solution filled in the syringe into a collector container filled with a poor solvent by extruding the spinning solution from the outlet of the injection needle having a predetermined diameter, wherein
the spinning solution injected into the poor solvent is solidified into fibers by interdiffusion of desorption of an organic solvent and penetration of the poor solvent, and the fibers solidified in the poor solvent are collected in cotton-wool like form by floating and depositing in the collector container without having adhesion between the fibers

2. The method according to claim 1, wherein the poor solvent is ethanol.

3. The method according to claim 1, wherein the poor solvent is water.

4. The method according to claim 1, wherein the calcium salt particles are β-TCP particles.

5. The method according to claim 4, wherein the β-TCP particles contain silver ions.

6. The method according to claim 4, wherein the β-TCP particles are silver ion incorporated β-TCP particles synthesized by substituting calcium in the crystal lattice of β-TCP with silver ions.

7. A cotton-wool-like bone regenerating material produced from an improved wet spinning process, the cotton-wool like bone regenerating material is produced by the process of:
preparing a spinning solution with a resin concentration of 10-20 wt % in which
calcium salt particles are dispersed by mixing the calcium salt particles of 50-80 wt % and PDLLGA resin of 50-20 wt % in a mixing container to form a mixture, and dissolving the mixture in acetone to form a dissolved mixture and stirring the dissolved mixture,
and
filling the spinning solution thus prepared in a syringe,
injecting the spinning solution filled in the syringe into a collector container filled with a poor solvent by extruding the spinning solution from the outlet of an injection needle having a predetermined diameter of the syringe, wherein
the spinning solution injected into the poor solvent is solidified into fibers by interdiffusion of desorption of an organic solvent and penetration of the poor solvent, and the fibers solidified in the poor solvent are collected in cotton-wool like form by floating and depositing in the collector container without having adhesion between the fibers.

8. The cotton-wool like bone regeneration material of claim 7, wherein the calcium salt particles are calcium phosphate particles.

9. The cotton-wool like bone regeneration material of claim 8, wherein the calcium phosphate particles are β-TCP particles.

10. The cotton-wool like bone regeneration material of claim 9, wherein the β-TCP particles are silver ion incorporated β-TCP particles synthesized by substituting calcium in the crystal lattice of β-TCP with silver ions.
